(19) United States
(12) Patent Application Publication
(10) Pub. No.: US 2017/0368097 A1
(43) Pub. Date: Dec. 28, 2017

(54) HAIR RESTORER PREPARATION OR FOOD SUPPLEMENT

(71) Applicant: Simone Hacki, Cham (CH)

(72) Inventor: Simone Hacki, Cham (CH)

(21) Appl. No.: 15/540,577

(22) PCT Filed: Dec. 30, 2015
(86) PCT No.: PCT/IB2015/060047

§ 371 Date: Jun. 29, 2017

(30) Foreign Application Priority Data
Dec. 30, 2014 (CH) CH02057/14

(51) Int. Cl. A61K 33/34; A23L 33/00; A61K 33/04; A61K 33/00; A61K 31/714; A61K 31/593; A61K 31/519; A61K 31/465; A61K 31/4188; A61K 31/375; A61K 31/355; A61K 31/20; A61K 31/198; A61K 31/197; A61K 31/122; A61K 9/00; A61K 33/06; A23L 33/155; A23L 33/16; A23L 33/175; A61K 33/26

(52) U.S. Cl. CPC A61K 33/34; A61K 33/26; A23L 33/30; A61K 33/04; A61K 33/00; A61K 31/714; A61K 31/593; A61K 31/519; A61K 31/465; A61K 31/4188; A61K 31/375; A61K 31/355; A61K 31/20; A61K 31/198; A61K 31/197; A61K 31/122; A61K 9/0053; A61K 33/06; A23L 33/155; A23L 33/40; A23L 33/16; A23L 33/175; A23V 2002/00

(57) ABSTRACT

The invention relates to a hair restorer preparation or food supplement (100, 100a-100c) for oral administration, comprising L-cysteine and zinc, wherein the hair restorer preparation respectively food supplement (100, 100a-100c) comprises copper, calcium, magnesium, silicium and cholecalciferol, and wherein the composing substances or substance compositions (S-Z) composing the hair restorer preparation respectively food supplement (100, 100a-100c) are determined in their dosage by means of tests and/or measurements and/or questionnaire evaluations (A-H) and are adaptable to the individual organic nutrients supply requirements of a female or a male customer (200, 200a-200d).

The present invention relates to a hair restorer preparation which is not intended for external application to the scalp but for oral administration and can at the same time be used as a food supplement.

There are some known hair restorer preparations for oral administration which take up the basic idea that an improved supply of the organism and the metabolism with the necessary acids, vitamins, minerals, trace elements, and so on, can perform a positive influence on the hair restoration.

For example, Priorin® has been on the market for a long time, a hair restorer preparation containing L-cysteine, a sulfur-containing proteinogenic amino acid, and a total extract of millet fruits and calcium pantothenate. The disadvantage of this hair restorer preparation, however, is that it covers only a very narrow spectrum of the substances required in its entirety, which can all be responsible for healthy hair growth. Furthermore, the composition is undifferentiated according to individual requirements.

Another hair restorer preparation, which was likewise developed for oral administration, is, for example, Amitamin® HairPlus. This hair restorer preparation is intended to cover a wider range of the necessary respectively beneficial substances by adding to the content, in addition to the already mentioned substances, yeast, biotin, vitamin E, amino acids, iron, zinc and others. But this preparation is also not customized.

In general, it is a disadvantage, in particular from the point of view of an economic efficiency ratio, when an undifferentiated “club” is thrown onto the market, which does not have a high percentage efficiency, and if it does, it is rather by chance.

The undifferentiation of such “ex-factory” hair restorer preparations, in the form of standard or monopreparations, is a disadvantage not only with respect to the presence of the substances or substance compositions which are usually necessary, which could be quite expensive, but also with regard to their dosage. These are held at a level which is still generally justifiable and correspondingly small, even though the female customer or the male customer would be dependent in particular on a specific substance or substance composition in a relatively increased dose, in relation to the remaining substances or substance compositions.

Hair restorer preparations which contain hormonally-active substances or substance compositions are also disadvantageous. For example, the active substance finasteride from the drug Propecia®, once started to be taken, it is necessary to maintain this dose for a lifetime. Otherwise, the situation worsens beyond the original initially stage. Furthermore, this hair restorer preparation is not suitable for women and attacks the hormonal balance with considerable negative side effects by suppressing the dihydrotestosterone level (DHT) in long term.

In addition, there is pantostine with the active substance alfastradiol, by means of which non-hormonal, but quasi-medically respectively chemically restoration of the hair roots will be attempted.

Pantovigar N is also orally administered and attempts to improve the nutrient supply by means of vitamins B and cysteine in such a way that the build-up of hair keratin will be promoted.

Further, there are capsules developed by Oxford Biolabs and marketed under the designation TRX2®, as a patent pending agent against Alopecia areata. This patent is not yet published at the time of writing this patent application and describes a combination of powerful metabolic stimulants (potassium, nicotinic acid and branched chain amino acids, like for example valine, leucine and isoleucine) with a natural energy-exciting substance (Carnipur™ tartrate). This combination, in turn, is designed to stop hair loss and promote hair growth at the molecular level. However, this agent is also not “tailor-made” to the female or male customer.

The object of the present invention is to provide a hair restorer preparation respectively combined nutritional supplement, which has a further improved basic composition and can be variable and/or adaptable to the individual needs of a female customer or a male customer, and in general, to its characteristics and to its application.

The solution to the task firstly consists, according to the invention, in the composition of a hair restorer preparation in a basic version which comprises the following basic substances or basic substance compositions:

L-cysteine

zinc

copper

calcium

magnesium

silicium

vitamin D3.

As already mentioned in the beginning, L-cysteine is a sulfur-containing proteinogenic amino acid and building block of the hair keratin and thus an important structural element for hair and nails. Thanks to the sulfur content, L-cysteine can bind heavy metal ions, which in turn can cause hair loss. L-cysteine, together with other amino acids (glutamine, glycine), is involved in the construction of glutathione, which is one of the most important antioxidants in the human body and is beneficial to the utilization of vitamin C and E. Glutathione also inhibits inflammatory reactions and stimulates the production of white blood cells and leukotrienes. The human liver can synthesise body-borne L-cysteine, but it must also be absorbed through the diet, with a daily requirement of about 1,400 mg. This need is often not covered because certain diseases such as cataracts or arthritis or other chronic diseases or even diets or stress or physical exertion increase it. As part of the food preparation process it is also an aggravating fact that, when the foods are for long time kept in the water or even boiled, the well-water-soluble L-cysteine is washed out.

On the other hand, the trace element zinc, is essentially involved in cell division in the human organism, in other words generally in growth and cell and skin regeneration, in insulin storage, in protein synthesis, in sperm production and in the immune system. Zinc is generally involved in hormone metabolism, has a positive effect on the central nervous system during stress, regulates the acid-base balance and helps to divert heavy metals. A lack of zinc can lead among other things to an increased hair loss, worse and to inflammation predispositioned skin and to a weakening of the immune system. Zinc deficiency is a problem especially in vegetarian nutritional forms, since zinc is mainly contained in meat, cheese, milk and eggs. The zinc contained in grain products is less readily absorbed from the intestine when the diet is low in protein. When zinc is given at the same time, it must be ensured that zinc intake in the intestine is worsened when co-administered with iron preparations.

The above-described basic composition of a hair restorer preparation according to the invention further comprises copper. The trace element copper is involved in the construction of collagen and thus ensures a tightening of skin tissue, connective tissue and nerve tissue and the blood vessels. Furthermore, the trace element copper is involved in the formation of melanin, the pigment for hair, skin and eyes. Gray hair can have a copper deficiency as a cause. Furthermore, copper is critically involved in the formation of endogenous antioxidants that delay the aging process.

In turn, calcium is no longer a trace element for the human body, but rather a quantity element, that is to say with more than 50 mg per kg of body weight. With a body weight of 1-1.1 kg, calcium is the most abundant mineral in the human organism. 99% of these are found in bones and teeth, but the body's calcium is also involved in muscle and nerve excitement, glycogen and amino acid metabolism, cell division, and the activation of some enzymes and hormones. The recommended daily intake for an adult person is very rough at about 1,000 mg. It is to be noted, that when the diet is particularly rich in protein, it is an increased demand for calcium.

Furthermore, magnesium is one of the essential substances because it is indispensable for all organisms. It must be daily administered to the body in sufficient quantities (approximately 300 mg) via food and drinking water and resorption takes place in the upper small intestine. The body of an adult contains about 20 g of magnesium, which is involved in approximately 300 enzyme reactions as enzyme component or coenzyme. In addition, free magnesium ions influence the potential of cell membranes and function as second messengers in the immune system. They stabilize the resting potential of excitable muscle and nerve cells and the cells of the autonomous nervous system. Magnesium is a muscle and connective tissue relaxant. Magnesium deficiency causes restlessness, nervousness, irritability, headache, lack of concentration, fatigue, general feeling of weakness, cardiac arrhythmias, muscle cramps and at least of course, that such a bad general condition in the nutrient supply will affect the hair growth.

Silicium is a main element of bone-forming cells, but also an important structural element for cartilage, connective and bone tissue, skin, hair and fingernails. Low hair-silicium values were observed during hair loss. Chronic stress with aluminum seems to impair the bioavailability of the silicium.

Vitamin D3 respectively cholecalciferol is necessary in the human organism, among others, for the fact that it can absorb larger amounts of calcium by ensuring the transport of calcium through the intestinal wall by the formation of a protein barrier. Vitamin D3 is the only vitamin in which the biologically active form is a hormone that plays a broad-spectrum role in the organism. It is important, among many others, on the bones and teeth development, but also to the immune system, by supporting the function of the white blood cells against infections. Due to the endemic light deficiency of modern societies, there is an insufficient organic synthesis of vitamin D3 in the skin, and therefore the German Society for Nutrition (DGE) gave a guideline for the daily vitamin D3 quantity of 20 μg for an adult, which is to be covered alimentarily. An aggravating fact is, that not only sunscreens, but also almost all of today's day creams contain UV-B and UV-A filters.

The dosage of these basic substances or basic substance compositions can be variable and adaptable to the individual organic nutrient supply requirements of the female or male customer, in consequence of the results of individual tests and measurements, but also according to the evaluation results of questionnaires.

The present invention is based on the following basic principles: It is, of course, possible for the head hair of a person to be assigned certain body functions or an organic “purpose”, but this respectively these are not of vital importance to the organism. For example, in the event of a death from frost, the blood supply to the extremities or the organs less important to the vital functions of the organism is discontinued, and only lastly the blood supply of the heart and brain. Similarly, the organism reacts with a persistent nutrient deficiency, and a prioritizing of the available nutrients takes place, first to the vital organs and parts of the body, and the hair growth is given a lower or even last priority.

A second basic idea is that fewer hair loss is genetically inherited, but rather caused by life habits and the ability to resorb certain nutrients better or worse, respectively to absorb respectively to recycle. Furthermore, a hair restorer preparation adapted according to the invention is based on the fact that about 80% of men are affected by hair loss and only about 38% of women. It is therefore necessary, at least with regard to the sex, to adapt the hair restorer preparation to the different prerequisites of the two sexes, or rather to further individual requirements.

Furthermore, a hair restorer preparation respectively food supplement according to the invention is based on the fact that the vitamin and nutrient content of the food has decreased by about 30% in our days, and at the same time, a more stressful and hectic lifestyle requires an increased intake of vital substances such as vitamins, trace elements and minerals, amino acids and quasivitamines.

Furthermore, findings on orthomolecular medicine are based on a hair restorer preparation according to the invention. Thereafter, an optimum metabolism takes place in the organism only when the high number of different vital substances is ensured in different and coordinated dosages. Already a single deficiency among the more than two dozen micronutrients required can slow down or even block the entire metabolism. As a result, the composition of a hair restorer preparation according to the invention is not only contented with individual substances or substance compositions and the minimum dosages recommended in the commonly cautiously recommended dosages, but preferably comprises many or all substances required by the body and without any variation of one or the other in very high dosage.

A further embodiment variant of a further hair restorer preparation according to the invention comprises, in addition to the basic substances or basic substance compositions listed in paragraph [0012], vitamin E. Vitamin E is a collective term for lipid-soluble substances with antioxidative action, most frequently in the form of tocopherols and tocotrienols occurrence. Vitamin E helps the immune system to remove oxidative influences from the environment, to divert toxic substances and destroy bacteria. It has also been observed that the demand for vitamin E increases with a diet containing an increased number of polyunsaturated fatty acids. The composition of a hair restorer preparation according to the invention can not lastly include vitamin E because it has been recognized that vitamin E promotes the blood circulation of the scalp and thereby strengthens the anchoring of the hair. Furthermore, vitamin E is required for the synthesis of various nutrients for the hair.

Furthermore, a further embodiment variant of a hair restorer preparation respectively combined food supplement according to the invention comprises vitamin C or ascorbic acid beyond the basic substances or basic substance compositions listed in paragraph [0012] and/or in paragraphs [0012] and [0025]. This is based on the knowledge that vitamin C helps in detoxification, for example is a potent antioxidant (“radical catcher”), acts as a stress management agent and thus provides more nutrients for hair growth. Vitamin C is also significantly involved in collagen production and also improves the structure of the hair.

A further embodiment variant of a further hair restorer preparation according to the invention, in addition to the base substances or base substance compositions disclosed up to this point, comprises biotin or vitamin H or vitamin B7. This is due to the fact that biotin is significantly involved in metabolic activation and cell growth, and a deficiency of this can, among other thirsts, lead to increased hair loss.

A further composition variant of a hair restorer preparation according to the invention also comprises L-methionine, an essential such as L-cysterine also sulfur-containing proteinogenic amino acid, from which the L-cysteine is formed. The L-methionine is involved in the synthesis of procollagen and is therefore important for hair formation.

A further preferred composition variant of a hair restorer preparation according to the invention also comprises pantothenic acid, trivially also called vitamin B5. It is the water-soluble vitamin from the series of B vitamins and is involved in the synthesis and breakdown of carbohydrates, fats, amino acids and in the synthesis of cholesterol, which in turn is needed for the formation of the steroid hormones. A deficiency can lead to fatigue, insomnia, depression, deaf and aching muscles, anemia, immunodeficiency and stomach pain and thus to such a bad general condition, that the required nutrient supply is detracted from the hair growth.

A further preferred embodiment variant of a hair restorer preparation respectively combined food supplement according to the invention, in addition to the disclosed basic substances or the basic substances plus one or a combination of the optional substances or plus all optional substances, comprises also iron. Iron is an essential trace element that is involved in blood formation and oxygen transportation, oxygen storage and electron transfer. Further, iron is a component of iron-sulfur complexes in many enzymes. It has been recognized that iron deficiency is common not only in women because of menstruation and births, but also in athletes. In the composition of a hair restorer preparation according to the invention or combined food supplements with iron, care is taken that a simultaneous intake of vitamin C significantly increases the resorption rate of iron, but milk products, coffee or black tea inhibit them.

In addition to the disclosed basic substances or the basic substances plus one or a combination of the optional substances or, in addition to all optional substances, vitamin B6 respectively the three precursors of the activated vitamin, pyridoxal phosphate, in particular, a further preferred embodiment variant of a hair restorer preparation respectively combined food supplement, namely Pyridoxine, pyridoxal and pyridoxamine. Vitamin B6 is essential for amino acid metabolism and acts as a coenzyme in about 100 enzymatic reactions. Vitamin B6 deficiencies are associated with loss of appetite, diarrhea, vomiting, dermatitis, growth disorders, anemia, nerve degeneration, convulsions at irregular intervals, and anxiety disorders. The need for vitamin B6 is directly proportional to protein gain and is recommended by the German Society of Nutrition (DGE) with a daily dose of 1.6 mg for women and 1.8 and for men.

A further preferred embodiment variant of a hair restorer preparation or combined food supplement according to the invention comprises, in addition to the disclosed basic substances or the basic substances plus one or a combination of the optional substances or, in addition to all optional substances, vitamin B12 or cobalamin named after its central atom cobalt. Vitamin B12 is important in the human organism for cell division and blood formation as well as the function of the nervous system. The human organism is not able to produce vitamin B12 itself and must therefore be fed through the food. This is above all guaranteed by the consumption of meat and offal, and must be increased by the consumption of dairy products and eggs or Nori algae. A deficiency of vitamin B12 can lead to diseases of the blood count and to myelitis an inflammation of the back or bone marrow. In recent years, moreover has been increased the evidence of a possible link between vitamin B12 deficiency and other pathologies, for example dementia and neuropathies. In general, low vitamin B12 concentrations in the blood serum are more frequently observed at elderly people. First signs of a vitamin B12 under-supply at adult persons can be tingling and feeling cold in hands and feet, exhaustion and weakness, concentration disorders and even psychoses. The daily minimum requirement for an adult is approximately 3.0 μg and for pregnant and breastfeeding women 3.5-4.0 μg.

A further preferred embodiment variant of a hair restorer preparation respectively combined food supplement according to the invention comprises, in addition to the disclosed basic substances or the basic substances plus one or a combination of the optional substances or, in addition to all optional substances, selenium. For all forms of life, selenium is an essential trace element, which protects the cell membranes from oxidative destruction, due to its high reactivity with oxygen. Furthermore, selenium and its compounds play an important role in the production of thyroid hormones. However, a positive effect of selenium is only observed in a very narrow and exact dosage range. There are studies that are suspected of causing type 2 diabetes by administering 200 μg of selenium per day. And in the case of employees in the electronics, glass and colour industries, who are exposed to daily doses of more than 400 μg, tread among others poisoning symptoms such as nausea and vomiting, nail changes, peripheral neuropathy, fatigue and of all things hair loss.

A further composition variant of a hair restorer preparation according to the invention also comprises niacin or nicotinic acid, which is another vitamin from the B complex. It is a crystalline solid found in all living cells and stored in the liver. It is of central importance for the metabolism of proteins, fats and carbohydrates and is important for the regeneration of skin, muscles, nerves and DNA. It is also intended to improve the blood circulation of the scalp and is required in an average energy requirement of an adult body in an amount of 6.6 mg to produce an energy quantity of 1,000 kilocalories for its cells, tissues and organs. Thus the need for women is 13 to 15 mg per day and for men 15 to 20 mg per day.

A further embodiment variant of a hair restorer preparation according to the invention comprises, in addition to the previous basic substances or also optional substances, also folic acid, which is also known under the trivial name vitamin B9 and a further light-sensitive vitamin from the B complex. Folic acid is involved in the synthesis of DNA building blocks and plays a decisive role in cell division. It is essential for the human organism and can not be produced by itself. Folic acid deficiency can lead, among other things, to anemia and favors the development of neural tube defects such as a spina bifida or anencephaly in embryonic development. The recommended daily dose according to RDA (Recommended Daily Allowance) or also according to the D-A-CH-Reference-Values for the nutrient supply is 200 μg. In some countries, for example also in Switzerland, an obligation to confine is designated by law.

A further preferred composition variant of a hair restorer preparation respectively combined food supplement according to the invention comprises, in addition to the basic substances or any combination of the basic substances with the optional substances, the coenzyme Q10, also known as ubiquinone 10. It is a quinone derivative, structurally related to vitamin K and vitamin E, and a carcinogenic substance that is partially absorbed through the food but is also produced in the body itself. Q10 is essential in converting in any human cell the energy from food into body energy (ATP). Q10 is an essential cofactor of the respiratory chain in the mitochondria of the cell and also serves as the most important lipophilic antioxidant in stress and degenerative processes as a free-radical catcher. A permanent Q10 deficiency is rare, but to avoid or compensate for a deficiency, most scientists recommend a daily dose as a dietary supplement of 30-200 mg per day.

A further preferred composition variant of a hair restorer preparation respectively combined food supplement according to the invention comprises, in addition to the basic substances or any combination of the basic substances with the optional substances, also omega-3 fatty acids. These are essential substances for human nutrition and can not be produced by the body itself. They are, among others, a reduction in cardiovascular risk, cancer prevention, inflammation inhibition, prevention of age-related degenerative diseases and dementia. In particular, the European Food Safety Authority (EFSA) and the German Society of Nutrition (DGE) recommend a dietary daily intake of 250 mg, in particular from the omega-3 fatty acids docosahexaenoic acid (DHA) and eicosapentaenoic acid (EPA). The omega-3 fatty acids are preferably admixed by means of microcapsules which are admixed with a preferably powdery composition or consistency of a hair restorer preparation respectively dietary supplement according to the invention and which contain the omega-3 fatty acids.

A preferred composition of a hair restorer preparation according to the invention further comprises guar flour to which the disclosed substances and substance compositions are bound. Guar flour is produced from the seeds of the guar bean and consists predominantly of long-chain carbohydrates, which are composed in a characteristic manner of the simple sugars mannose and galactose. The long-chain compounds can bind very large amounts of water. This and the slimy consistency are presumably the cause for the observation that the substances and substance compositions of a hair growth preparation respectively food supplement according to the invention are transported much better into the small intestine. In contrast to the stomach or duodenal intestine, an almost 100% increased uptake of the substances and substance compositions takes place in the small intestine, without undesirable interactions. In addition, the guar flour has the positive side effect that it has an intestinal caring effect. In this way, the guar flour fulfills the function of a retarder according to the invention, which allows the absorption of the nutrients to take place where it is most effective.

In the case of possible allergic reactions to the guar flour, the composition of a preferred hair restorer preparation according to the invention described above is preferably selected from the locust bean gum.

From all these findings and inventive considerations, it results an optional and exemplified complete version of a hair restorer preparation respectively food supplement according to the invention, for example for an adult human being with the following exemplified, not necessarily cumulative substances or substance compositions and with the following likewise preferred daily doses and daily doses-ranges:

| | Preferred | Range |
|---|---|---|
| Nutrient | daily dose | daily dose |
| Vitamin | | |
| biotin | 5.0 mg | 0.1-40.0 mg |
| folic acid | 200.0 μg | 100.0-2,000.0 μg |
| natural carotenoids | 8.0 mg | 1.0-160.15 mg |
| thereof alpha-carotene | 70.0 μg | 1.0-100,000.0 μg |
| thereof cryptoxanthin | 15.0 μg | 1.0-50.0 μg |
| thereof lutein | 6.0 mg | 1.0-15.0 mg |
| thereof beta-carotene | 1.9 mg | 1.0-45.0 mg |
| thereof zeaxanthin | 15.0 μg | 1.0-100.0 μg |
| Natural vitamin E | 300.0 mg | 1.0-1,000.0 mg |
| thereof alpha-tocopherol | 262.0 mg | 85-95% |
| thereof gamma-tocopherol | 30.0 mg | 5-15% |
| niacin | 50.0 mg | 1.0-500.0 mg |
| pantothenic acid | 10.0 mg | 1.0-1,000.0 mg |
| Vitamin A (retinoids) | 1.0 mg | 1,000-40,000 IE |
| Vitamin B₁ (thiamine) | 5.0 mg | 1.0-200.0 mg |
| Vitamin B₂ (riboflavin) | 5.0 mg | 1.0-100.0 mg |
| Vitamin B₆ | 10.0 mg | 1.0-480.0 mg |
| Vitamin B₁₂ | 35.0 μg | 1.0-1,000.0 μg |
| Vitamin C | 1,500.0 mg | 1.0-10,000.0 mg |
| Vitamin D3 | 7 μg | 1.0-100.0 μg |
| trace elements | | |
| chrome | 50.0 μg | 1.0-300.0 μg |
| iron | 2.5 mg | 1.0-100.0 mg |
| iodine | 50.0 μg | 1.0-1,000.0 μg |
| copper | 2.0 mg | 0.1-5.0 mg |
| manganese | 5.0 mg | 1.0-50.0 mg |
| molybdenum | 50.0 μg | 1.0-1,000.0 μg |
| selenium | 200.0 μg | 1.0-400.0 μg |
| vanadium | 50.0 μg | 1.0-200.0 μg |
| zinc | 25.0 mg | 1.0-100.0 mg |
| minerals | | |
| calcium | 800.0 mg | 1.0-1,500.0 mg |
| potassium | 220.0 mg | 1.0-5,000.0 mg |
| magnesium | 400.0 mg | 1.0-1,500.0 mg |
| silicium | 26.0 mg | 1.0-50.0 mg |
| quasi-vitamins | | |
| a-lipoic acid | 20.0 mg | 1.0-1,000.0 mg |
| choline | 40.0 mg | 1.0-10,000.0 mg |
| inositol | 30.0 mg | 1.0-3,000.0 mg |
| L-carnitine | 250.0 mg | 1.0-3,000.0 mg |
| p-aminobenzoic acid (PABA) | 10.0 mg | 1.0-4,000.0 mg |
| coenzyme Q10 (ubiquinone-10) | 100.0 mg | 1.0-600.0 mg |
| omega-3 fatty acids | 250.0 mg | 100.0-600.0 mg |
| amino acids | | |
| L-cysteine | 1,000.0 mg | 1.0-2,000.0 mg |
| L-methionine | 400.0 mg | 1.0-5,000.0 mg |
| gamma-aminobutyric acid (GABA) | 1.0 mg | 0.1-5,000.0 mg |
| plant extracts | | |
| silymarin | 150.0 mg | 1.0-220.0 mg |
| citrus bioflavonoids | 200.0 mg | 1.0-400.0 mg |
| fibers | | |
| guar | 200.0 mg | 1.0-500.0 mg |

The following are considered as tests and measurements: blood, urine, creatinine, saliva, hair, stoole and breath tests, the last one in particular for determining the acid content or the PH value in the exhaled breathing air. The following variants of a hair restorer preparation according to the invention described below are based, among others, on the fact that an over-acidification of the human organisms is widely spread and thus inhibits the intake of many vitamins, trace elements, minerals and amino acids.

The above-mentioned blood test is preferably composed of the following single tests: blood count, blood reduction, thyroid and renal function parameters, iron in serum, iron binding capacity, zinc and selenium from whole blood, calcium from serum and whole blood, transaminases and immunoglobulin E (IgE)-levels.

As a questionnaire or even as a guide for the recording of the customer data, it is possible, for example, to query the sex, the body size, the weight, the age, the health condition, the taking of medicine and other eating, drinking and living habits. Furthermore, for example, a query is also made about the customer preference for his hair restoration.

According to the invention, the basic dosage of the hair restorer preparation is adjusted in consequence of the tests and measurements, but optionally also in consequence of only the answers to the questionnaire. Thus, for example, it is intended to place a man on a daily dose of 25.0 mg or even 30.0 mg of zinc, different from a woman, to whom 20.0 mg of zinc are designated. Zinc dosage will be increased for men, as they have increased demand for spermatozoa compared to women.

Furthermore, it is possible to designate for a woman a daily dosage of 3.0 mg of gamma-aminobutyric acid, different from a man with only 1.0 mg designated. The gamma-aminobutyric acid GABA is increased for women because they can suffer from the premenstrual syndrome and this can be eased by the administration of GABA. In addition, unlike the men, women have a lower protein reserve. That's why they need more GABA. The GABA has a positive, strengthening effect in combination with vitamin B6 and L-cysteine. The man, on the other hand, often has a or a partial bald head and thus has an increased demand for L-cysteine, which is why he requires less GABA. For those who consume alcohol more than three times per week, GABA is increased because GABA can reduce the longing for alcohol by its nerve-calming effects.

Furthermore, with regard the different sexes, it can also be designated according to the invention, for a man only 1.0 mg of iron instead of, for example, 2.5 mg for a woman. The iron designation can be somehow reduced in men because it has no iron loss over menstruation and consumes more meat than a woman from its average food habits.

Concerning the body size and the weight, it is also designated according to the invention to add to the composition 13.0 mg of L-methionine, per kg of body weight.

Depending on the age of the customer, an age-graded quantity of vitamin B12 is designated, which can basically move in a range of 1.0-1,000.0 μg, but preferably in a range of 20.0 μg to 50.0 μg and is preferably 35.0 μg. Furthermore, preferably 20.0 μg at an age of 18-35 years, 35.0 μg at an age of 35-60 years, and 50.0 μg at an age of 60-99 years.

In the case of an existing thyroid gland dysfunction, the amount of iodine can be increased to 200.0 μg.

In the case of an already existing cancer disease, the preferred amount of 10.0 mg vitamin B6 indicated in the table can be increased to 25.0 mg. Furthermore, the amount of 0.2 mg folic acid to 0.4 mg. Q10 can range from 100.0 mg to 150.0 mg and GABA from 1.0 mg to 2.0 mg. In addition, vitamin B12 can be increased from 35.0 μg to 50.0 μg.

In the case of diabetes, the L-cysteine can be reduced from its preferred amount and stated in the table from 1000 mg to 400.0 mg. In addition, vitamin B1 can also be increased from 5.0 mg to 50.0 mg and vitamin B6 from 10.0 mg to 50.0 mg. Furthermore, chromium from 50.0 μg to 200.0 μg.

In the case of stress, psychological or physical, the vitamin C can be increased from 1,500.0 mg to 2,000.0 mg and folic acid can be increased from 200.0 μg to 800.0 μg.

If a female customer drives sports more than three times a week, the zinc can be increased from the quantity indicated in the table as being preferred from 25.0 mg to 30.0 mg. If this activity is the same for a man, it can be increased to 40.0 mg.

If the female customer drinks alcohol more than three times per week, zinc can be increased from the preferred quantity indicated in the table from 25.0 mg to 30.0 mg and GABA can be increased from 1.0 mg to 3.0 mg. The dose of niacin can also be increased from 50.0 mg to 300.0 mg.

In the case of a customer who regularly smokes, a zinc deficiency can occur through the cigarette smoke, which in turn may lead to a copper excess or even a copper poisoning. As a result, for smokers, the copper can be reduced from the preferred amount shown in the table from 2.0 mg to 1.0 mg. In addition, in the case of smokers, the vitamin C can be increased from 1,500.0 mg to 2,000.0 mg. Furthermore folic acid from 0.2 mg to 0.4 mg, vitamin B6 from 10.0 mg to 25.0 mg and vitamin B12 from 35.0 μg to 40.0 μg. In addition, selenium can also be increased from 200.0 μg to 250.0 μg.

A hair restorer preparation respectively food supplement according to the invention can optionally be accompanied or preceded by a deacidification of the intestinal tract or of the organism. At the same time, silicium, zinc and iron can be added, the latter two trace elements preferably in chelated form.

If necessary, a prior intestinal rehabilitation can be designated for an optimal absorption of the substances and substance compositions.

Basically, however, it would also be possible to administer a hair restorer preparation respectively combined food supplement according to the invention also intramuscularly or subcutaneously or intravenously and possibly with regard to certain substances by depot injection.

A hair restorer preparation according to the invention, as just described, and as yet going to be described in the further embodiment variants in the present application, is generally and likewise suitable as a food supplement, if not already mentioned in the respective text passage. It has been observed that a hair restorer preparation respectively combined food supplement according to the invention is good for a healthy and strong nail growth as well as for a healthy head scalp. It also has a positive effect on diabetes melitus and against myocardial infarction.

A hair restorer preparation respectively food supplement according to the invention is preferably produced or provided in a generalized basic version with average dosages for women and a generalized basic version with average dosages for men. After evaluation of the tests and measurements as well as of the questionnaires, this basic version can be supplemented with the necessary difference substances or difference dosages to the individual tailor-made hair growth preparation or food supplements according to the invention.

The disclosed different embodiment variants of a hair growth preparation or combined food supplements according to the invention can be combined with one another as desired with regard to the optional substances.

The present application discloses a method for customer-specific adjustment of a food supplement as disclosed, comprising the following basic procedure steps:

a)—providing a general version of the food supplement with an average dosage for women;
b)—providing a general version of the food supplement with an average dosage for men;
c)—answering questionnaires by a female customer or by a male customer;
d)—if necessary, carry out tests and measurements;
e)—determining by means of the questionnaire evaluations and of the tests and measurements the difference substances and difference dosages;
f)—selecting the general food supplement version for women in the case of a female customer;
g)—selecting the general food supplement version for men in the case of a male customer;
h)—adapting the selected food supplement version to the individual female customer or to the individual male customer by adding the determined difference substances and difference dosages.

A hair restorer preparation according to the invention respectively food supplement according to the invention brings the following advantages, many of which are demonstrated by probands:

The hair restorer preparation respectively food supplement is individually tailor-made to the customer.

Vitality deficiencies are determined.

All necessary vital substances are covered, with top dosages for the missing ones.

It is well tolerated.

It is equally suitable as a hair restorer preparation as well as a food supplement.

It increases health and well-being.

Probands feel less tired and have more energy.

The hair growth is stimulated, both the number of hairs, as well as their strength and health.

The hair becomes luster.

Thin hair becomes fuller.

Brittle hair becomes supple.

Scaly scalp skin is restored.

The speed of the hair growth is accelerated within an anagen phase.

Further or advantageous embodiments of a hair restorer preparation according to the invention form the subject matter of the dependent claims.

The reference list is an integral part of the disclosure.

By means of a drawing, a partial view of the invention is explained in more detail, in a symbolic and exemplified manner. It presents schematic and exemplified representations and is not true to scale, even in the relation of the individual components to each other. Identical reference symbols mean the same object or component, reference characters with different indices indicate functionally equivalent or similar objects or components.

In this connection,

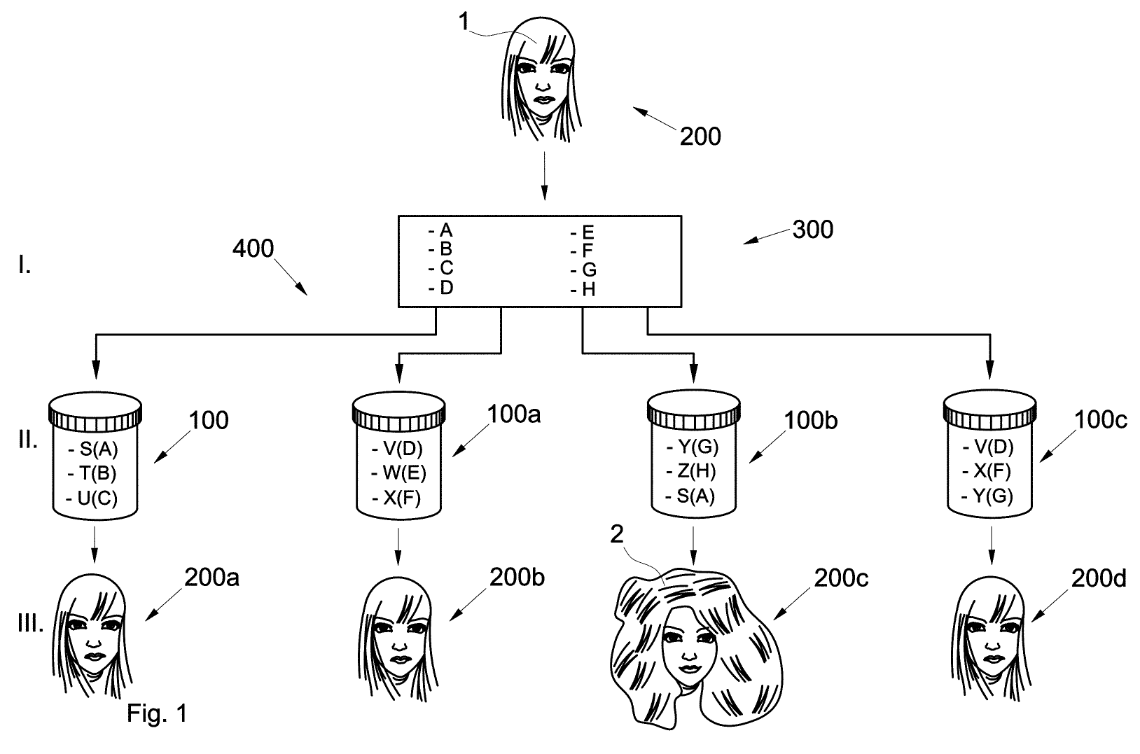

FIG. 1 shows a schematic representation of an exemplified customer adjustment of a hair restorer preparation respectively food supplement according to the invention.

In FIG. 1 it is shown, by way of example, a female customer 200 who is seeking help because of a sparse hair growth 1. A first phase I is a customer acquisition or requirement determination 300, comprising tests, measurements, and questionnaire evaluations A-H.

A second phase II of a preparation adjustment 400 represents that a first hair restorer preparation 100 comprises substances or substance compositions S-U which have been selected in consequence of tests A-C. By way of example, a further second hair restorer preparation 100a is shown in which substances or substance compositions V-X were selected in consequence of tests D-F. Further exemplified is a third hair restorer preparation 100b in which a substance or substance composition Y is selected in consequence of the test G as well as a substance or substance composition Z in consequence of the test H and the substance or substance composition S in consequence of the test A. A fourth and last exemplified embodiment variant of a hair restorer preparation 100c comprises the substance or substance composition V in consequence of the test D, the substance or substance composition X in consequence of the test F and the substance or substance composition Y in consequence of the test G.

A third phase III shows, symbolically and by way of example, that the female customer 200 from phase I has indeed become a “changed” female customer 200a by taking the hair restorer preparation 100, but has not yet made a great success with regard to improved hair growth. The same applies correspondingly to the hair restorer preparations 100a and 100c, or to the female customers 200b and 200d. In the case of the hair restorer preparation 100b, on the other hand, the female customer 200 has become a female customer 200c with a strong and voluminous hair growth 2. A time axis of phases I-III takes in each case several months.

### REFERENCE LIST

1—sparse hair growth
2—strong, voluminous hair growth
100, 100a-100c—hair growth preparation, food supplement
200, 200a-200d—female customer
300—customer acquisition, requirement determination
400—preparation adjustment
I—first phase
II—second phase
III—third phase
A-H—tests, measurements, questionnaire evaluations
S-Z—substance or substance composition

1. Hair restorer preparation or respective food supplement for oral administration, comprising L-cysteine and zinc, characterized in that the hair restorer preparation respectively food supplement comprises copper, calcium, magnesium, silicium and cholecalciferol.

2. Hair restorer preparation or respective food supplement as claimed in claim 1, wherein the substances or substance compositions (S-Z) composing the hair restorer preparation or food supplement are determined in their dosage by means of tests and/or measurements and/or questionnaire evaluations (A-H) and are adaptable to the individual organic nutrients supply requirements of a female or a male customer.

3. Hair restorer preparation or respective food supplement according to claim 1 wherein the hair restorer preparation respectively food supplement comprises natural vitamin E in the form of alpha-tocopherol and gamma-tocopherol.

4. Hair restorer preparation or respective food supplement according to claim 1 wherein the hair restorer preparation respectively food supplement comprises ascorbic acid.

5. Hair restorer preparation or respective food supplement according claim 1 wherein the hair restorer preparation respectively food supplement comprises biotin.

6. Hair restorer preparation or respective food supplement according to claim 1 wherein, characterized in that the hair restorer preparation respectively food supplement comprises L-methionin.

7. Hair restorer preparation or respective food supplement according to claim 1 wherein the hair restorer preparation respectively food supplement comprises pantothenic acid.

8. Hair restorer preparation or respective food supplement according to claim 1 wherein the hair restorer preparation respectively food supplement comprises iron.

9. Hair restorer preparation or respective food supplement according to claim 1 wherein the hair restorer preparation respectively food supplement comprises pyridoxine and/or pyridoxal and/or pyridoxamine.

10. Hair restorer preparation or respective food supplement according to claim 1 wherein the hair restorer preparation respectively food supplement comprises cobalamin.

11. Hair restorer preparation or respective food supplement according to claim 1 wherein the hair restorer preparation respectively food supplement comprises selenium.

12. Hair restorer preparation or respective food supplement according to claim 1 wherein the hair restorer preparation respectively food supplement comprises nicotinic acid.

13. Hair restorer preparation or respective food supplement according to claim 1 wherein the hair restorer preparation respectively food supplement comprises folic acid.

14. Hair restorer preparation or respective food supplement according to claim 1 wherein the hair restorer preparation respectively food supplement comprises ubiquinone-10.

15. Hair restorer preparation or respective food supplement according to claim 1 wherein the hair restorer preparation respectively food supplement comprises omega-3 fatty acids which are admixed to a powdery composition of the hair restorer preparation respectively food supplement in the form of microcapsules containing the omega-3 fatty acids.

16. Hair restorer preparation or respective food supplement according to claim 1 wherein the hair restorer preparation respectively food supplement comprises guar flour.

17. Hair restorer preparation or respective food supplement according to claim 1 wherein the hair restorer preparation respectively food supplement comprises natural carotenoids, retinoids, thiamine, riboflavin, chromium, iodine, manganese, molybdenum, vanadium, calcium, potassium, α-lipoic acid, choline, inositol, L-carnitine, p-aminobenzoic acid, gamma-aminobutyric acid, sylimarin and citrus bioconflavonoids.

18. Method for the customer-specific adaptation of a food supplement according to claim 1 wherein the following process steps are carried out:
a)—providing a general version of the food supplement with average dosages for female;
b)—providing a general version of food supplements with average dosages for male;
c)—answering questionnaires (A-H) by a female customer or a male customer;
d)—if necessary, carrying out tests and measurements (A-H);
e)—determining by means of the questionnaire evaluations (A-H) and the tests and measurements (A-H) of the difference substances and difference dosages;
f)—selecting the general food supplement version in the case of a female customer;
g)—selecting the general food supplement version in the case of a male customer;
h)—adapting the selected food supplement version to the individual female customer or to the individual male customer by adding the determined difference substances and difference dosages.
